(19) United States
(12) Patent Application Publication
(10) Pub. No.: US 2017/0368250 A1
(43) Pub. Date: Dec. 28, 2017

(54) Wearable Ultrafiltration Devices Methods and Systems

(71) Applicant: The Trustees of Columbia University in the City of New York, New York, NY (US)

(72) Inventors: Robert J. Von Gutfeld, New York, NY (US); Arthur Autz, Douglaston, NY (US); Edward F. Leonard, Bronxville, NY (US)

(73) Assignee: The Trustees of Columbia University in the City of New York, New York, NY (US)

(21) Appl. No.: 15/615,541

(22) Filed: Jun. 6, 2017

Related U.S. Application Data
(60) Provisional application No. 62/346,404, filed on Jun. 6, 2016.

(51) Int. Cl. A61M 1/34; A61M 39/10; A61M 39/28; A61M 39/22; A61M 1/36

(52) U.S. Cl. CPC A61M 1/3479; A61M 39/10; A61M 39/28; A61M 1/3626; A61M 2205/8206; A61M 2209/088; A61M 2039/229

(57) ABSTRACT

A wearable ultrafiltration apparatus is provided. The apparatus can include a first dialyzer for filtering a patient's blood along a first fluid path and a second dialyzer for filtering the patient's blood along a second fluid path. The apparatus can also include a valve being positionable in a first position for directing the patient's blood along the first fluid path. The valve can also be positioned in a second position for directing the patient's blood along the second fluid path. When the valve is in the first position, blood can flow along the first fluid path and prevent blood from flowing along the second fluid path. When the valve is in the second position, blood can flow along the second fluid path and prevent blood from flowing along the first fluid path. When the valve is in the first position, the second dialyzer can be idle and capable of being serviced or replaced and when the valve is in the second position, the first dialyzer can be idle and capable of being serviced or replaced. Therefore, when a dialyzer fouls, blood can be directed to the other dialyzer while the fouled dialyzer is being serviced or replaced.

### RELATED APPLICATIONS

This application claims priority to U.S. Provisional Patent Application Ser. No. 62/346,404 filed on Jun. 6, 2016, the entire disclosure of which is hereby incorporated by reference.

### BACKGROUND

### Field of the Disclosure

The present disclosure relates to a wearable device for removing unwanted waste products from a person's blood. More particularly, the present disclosure relates to wearable ultrafiltration devices, methods, and systems.

### Related Art

When treating patients with inadequate kidney function, it is highly desirable to remove excess metabolites, especially water, in a slow, continuous fashion, thus stabilizing blood volume and pressure. A wearable removal system permits continuous processing without confining the patient to the clinic. However, wearable removal systems or metabolite-removing devices, such as dialyzers or ultrafilters, have short lifetimes due to continuous processing. This leads to limited treatment times out of the clinic and could require the patient go back to the clinic frequently for a new device.

Therefore, there exists a need for a device, system, and/or method for transferring the blood removal function from one such device to a successor device by the patient in a coordinated, complete and safe manner. Such a feature would remove device failure as a major cause of limited treatment times out of the clinic, as well as providing other benefits.

### SUMMARY

A wearable ultrafiltration apparatus is provided. The apparatus can include a first dialyzer for filtering a patient's blood along a first fluid path and a second dialyzer for filtering the patient's blood along a second fluid path. The apparatus can also include a valve that is positionable in a first position for directing the patient's blood along the first fluid path. The valve can also be positioned in a second position for directing the patient's blood along the second fluid path. When the valve is in the first position, blood can flow along the first fluid path and prevent blood from flowing along the second fluid path. When the valve is in the second position, blood can flow along the second fluid path and prevent blood from flowing along the first fluid path. When the valve is in the first position, the second dialyzer can be idle and can be serviced or replaced, and when the valve is in the second position, the first dialyzer can be idle and can be serviced or replaced. Therefore, when a dialyzer fouls, blood can be directed to the other dialyzer while the fouled dialyzer is being serviced or replaced.

In a first embodiment of the apparatus the valve can include a first pinch valve for directing blood along the first fluid path and a second pinch valve for directing blood along a second fluid path. The first fluid path and the second fluid path can converge at a Y connector before the patient's blood is returned to the patient. The first fluid path can include a third pinch valve for directing blood flow to the Y connector and the second fluid path can include a fourth pinch valve for directing blood flow to the Y connector. When the valve is in the first position, the first and third pinch valves can be in an open position to direct blood flow along the first fluid path and the second and fourth pinch valves can be in a closed position to prevent blood flow along the second fluid path. When the valve is in the second position, the second and fourth pinch valves can be in an open position for directing blood flow along the second fluid path and the first and third pinch valves can be in a closed position for preventing blood flow along the first fluid path. The first dialyzer can include a first port for filtrate water disposal and the second dialyzer can include a second port for filtrate water disposal. A battery operated pump can direct the patient's blood to the apparatus. The apparatus can include a waste container attached to the first port and the second port by a common outlet. The waste can be collected without the need for a waste pump. The first dialyzer and the second dialyzer can be microtubular membrane filters of a type used for hemofiltration.

In a second embodiment, the valve can include a rotatable rod assembly. The rod assembly can include a rod and a handle for rotating the rod from a first position to a second position. A first edge of the rod can direct the patient's blood to the first dialyzer along the first fluid path. When the rod is in the second position, the first edge can be positioned in a second direction for directing the patient's blood to the second dialyzer along the second fluid path. A second edge of the rod directs the blood from the first dialyzer when the rod is in the first position or the second dialyzer when the rod is in the second position. The rod can be rotated along a longitudinal axis.

In a third embodiment, the valve can include a spigot handle having a shaft operable to a first position and a second position. The first fluid path can include a first port for directing blood to the first dialyzer and a second port for receiving blood from the first dialyzer. The second fluid path can include a first channel for directing blood to the second dialyzer and a second channel for receiving blood from the second dialyzer. When the spigot handle is in the first portion, blood flows through the first fluid path and when the spigot handle is in the second position, blood flows through the second fluid path.

A method of providing ambulatory ultrafiltration to a patient is also provided. The method includes the step of fitting an wearable ultrafiltration apparatus to a patient. The method further includes ultrafiltering said patient between dialytic treatments by passing the patient's blood through a first fluid path. The ultrafiltering step can also include detecting a fouling of a dialyzer in the first fluid path and closing the first fluid path using a valve. The ultrafiltering step can also include replacing the fouled dialyzer in the first fluid path with a new dialyzer. The method can further include operating said valve to allow flow through a second fluid path to permit flow of blood through a second dialyzer.

### BRIEF DESCRIPTION OF THE DRAWINGS

The foregoing features of the disclosure will be apparent from the following Detailed Description, taken in connection with the accompanying drawings, in which:

FIG. 1 is a diagram illustrating a first embodiment of the wearable ultrafiltration device of the present disclosure;

FIGS. 2A-2D are diagrams illustrating a second embodiment of the wearable ultrafiltration device of the present disclosure; and

FIGS. 3A-3D are diagrams illustrating a third embodiment of the wearable ultrafiltration device of the present disclosure.

### DETAILED DESCRIPTION

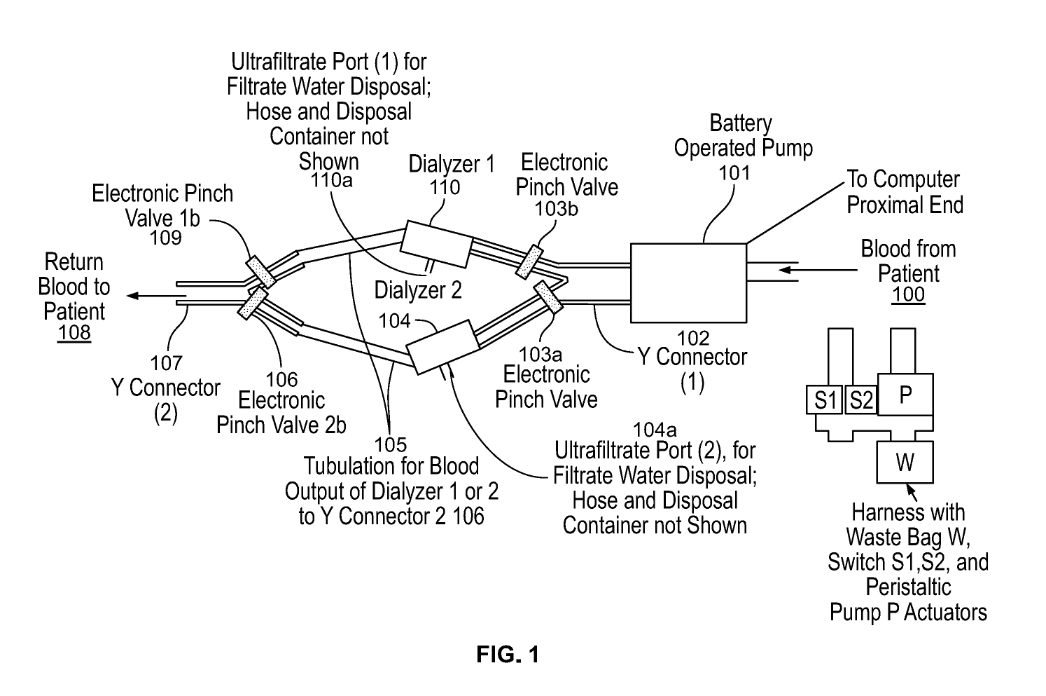

The present disclosure relates to wearable ultrafiltration devices, methods, and systems, as discussed in detail below in connection with FIGS. 1-3D.

FIG. 1 is a diagram illustrating a first embodiment of the wearable ultrafiltration device of the present disclosure. FIG. 1 shows blood from a patient being drawn by a battery operated pump 101 and distributed to one of two ultrafiltration devices (e.g., dialyzers). In particular, blood can be distributed to a dialyzer 110 or a dialyzer 104. It should be obvious to those skilled in the art that the device could easily be configured to accommodate three or more dialyzers. The dialyzers can be attached to a Y connector 102 where a pinch valve 103a and a pinch valve 103b can direct blood flow to the dialyzer 104 and the dialyzer 110. If it is needed for blood flow to be directed to the dialyzer 104, then pinch valve 103a can be in the open position and the pinch valve 103b can be in the closed position. If it is needed for blood flow to be directed to the dialyzer 110, then pinch valve 103b can be in the open position and the pinch valve 103a can be in the closed position. Once the blood is directed to either the dialyzer 104 or the dialyzer 110, it is then directed to a second Y connector 107. If blood is directed to the dialyzer 104, then a pinch valve 106 can be in the open position. If blood is directed to the dialyzer 110, then a pinch valve 109 can be in the open position. From the Y connector 107, blood returns to the patient through flow path 108.

As can be seen, the arrangement in FIG. 1 defines two fluid flow paths, one for the dialyzer 104 and another for the dialyzer 110. Initially, one fluid flow path and one dialyzer can be selected for filtering a patient's blood. For example, dialyzer 104 can be selected initially and pinch valve 103a can be open and pinch valve 103b can be closed. Moreover, the pinch valve 106 can be open and the pinch valve 109 can be closed. This directs blood flow to dialyzer 104 and not to dialyzer 110. When the dialyzer 104 begins to foul, blood can be directed to the flow path for the dialyzer 110. To switch flow paths, pinch valves 103a and 106 are closed and pinch valves 103b and 109 are open. This directs blood flow to the dialyzer 110 (which is unused in this example) and away from the dialyzer 104 (which has fouled in this example). While the dialyzer 110 is filtering blood and that fluid flow path is selected, the dialyzer 104 can be serviced or replaced so that when the dialyzer 110 begins to foul, the fluid flow path can be switched back to direct blood flow to the dialyzer 104. It should be noted that any fluid flow path can be selected first, and the other fluid flow path can be selected by opening the corresponding pinch valves and closing the pinch valves for the initially selected fluid flow path. The water taken out of the blood from the two dialyzers empties freely into a receptacle via dialyzer ports 104a and 110a. The output blood, 108, with a prescribed amount of its water content removed by way of the dialyzers is returned to the patient. The sequence of operations of the valves as described can be determined by a microprocessor.

It should be noted that in this embodiment, a patient or the patient's caretaker or other user can control whether the pinch valves remain open or closed. Therefore, such a person can control the fluid path and the dialyzer which is filtering the patient's blood. Moreover, in all embodiments, such a person can control any feature of the present disclosure (e.g. a valve) to change the direction of the fluid flow path from one dialyzer to another.

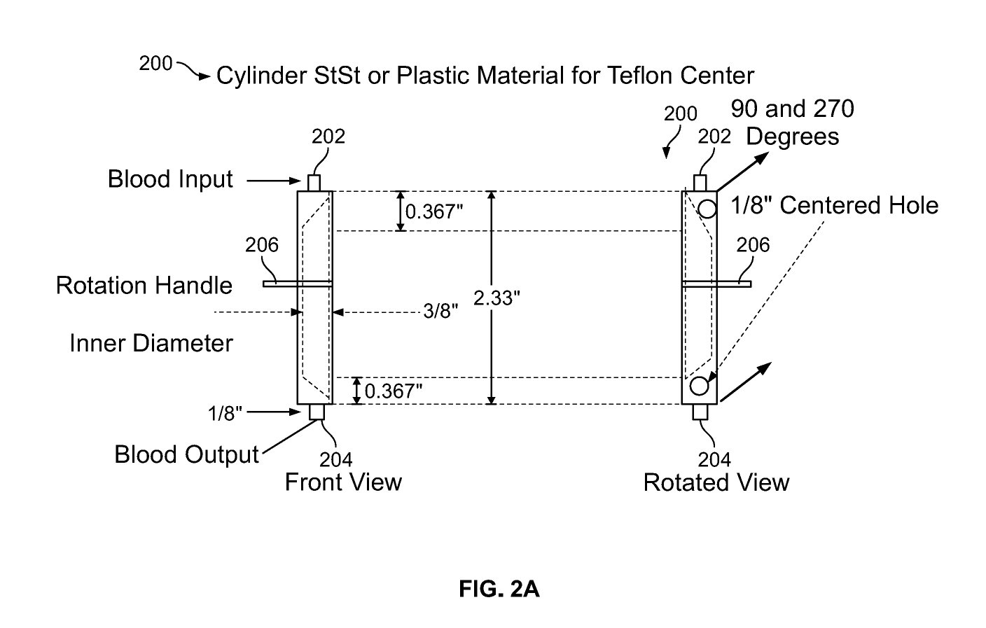

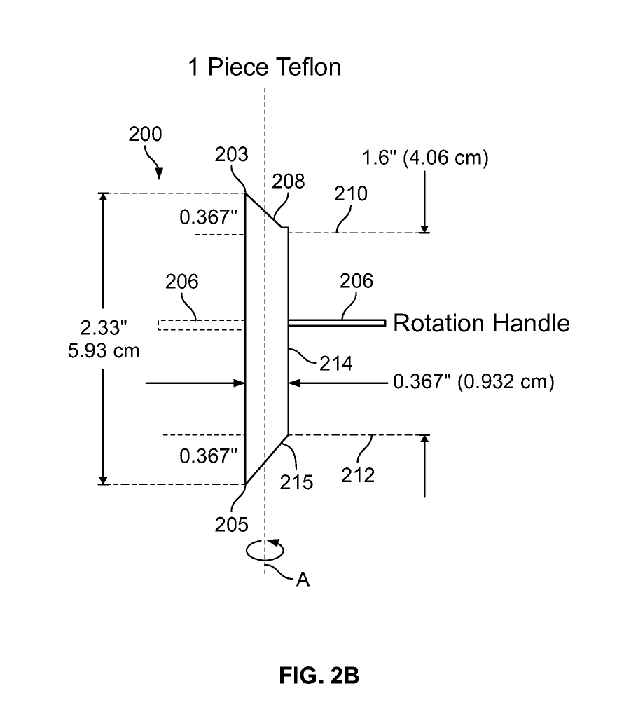

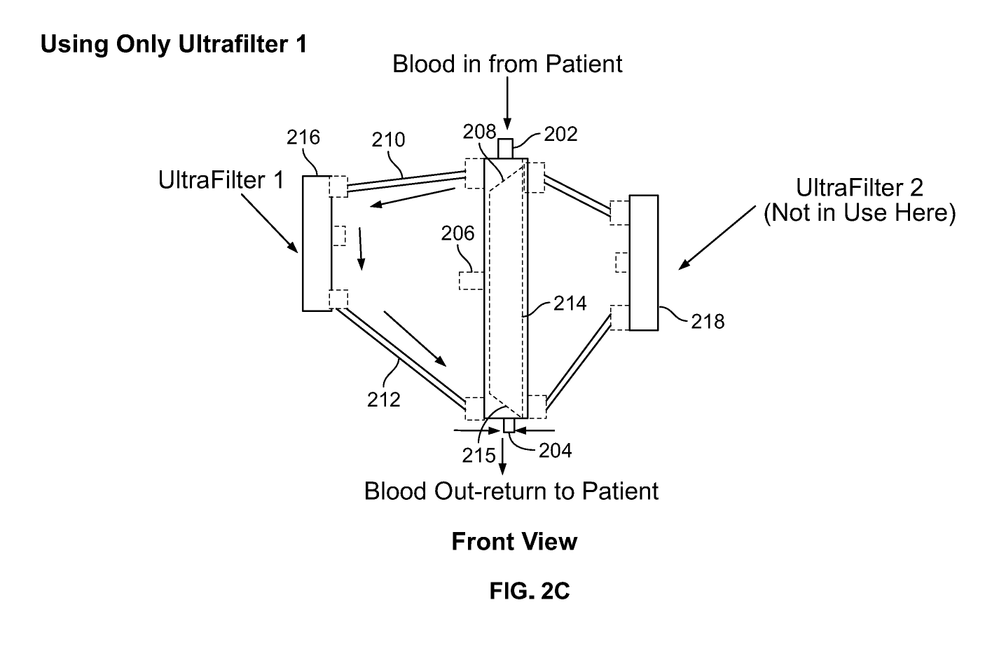

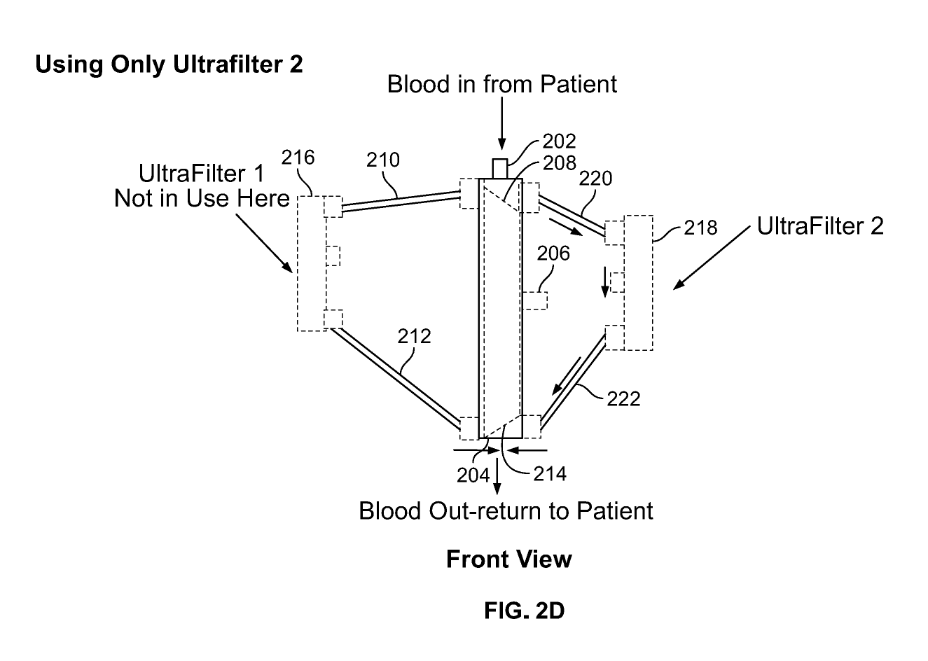

FIGS. 2A-2D are diagrams illustrating a second embodiment of the wearable ultrafiltration device of the present disclosure. The measurements shown in the drawings are for illustration purposes only and does not limit the scope of the present disclosure. FIG. 2A shows a front view and a rotated view, respectively, of a valve 200 for directing blood flow. The valve 200 includes a rod 214 (see FIG. 2b) as well as a blood input port 202, a blood output port 204, and a rotation handle 206. The rotation handle 206 changes the orientation of the rod 214 to direct blood flow from a first dialyzer to a second dialyzer. The rotation handle 206 is in a first position in the front view of FIG. 2A, and in a second position in the rotated view in FIG. 2A. When the rotation handle 206 is in the first position, blood can be directed to a first dialyzer and when the rotation handle 206 is in the second position, blood can be directed away from the first dialyzer and to a second dialyzer. As discussed with respect to the first embodiment in FIG. 1, once the first dialyzer begins to foul, the rod 214 can be rotated by the rotation handle 206 to direct blood flow away from the first dialyzer which has fouled, to the second dialyzer which can be unused. While the second dialyzer is filtering blood, the first dialyzer can be replaced or serviced so that when the second dialyzer fouls, the rod 214 can be rotated by the rotation handle 206 to direct blood flow away from the now fouling second dialyzer and to the newly-serviced or repaired/replaced first dialyzer (path).

FIG. 2B shows a second front view of the valve 200 for directing blood flow. When blood reaches the rod 214 via a first port proximal to end 203, the blood can travel along a chamfered edge 208 to a flow path 210, which directs the blood to a first dialyzer. After the first dialyzer filters the blood, it can travel back along a path 212 and along a chamfered edge 214 and away from the rod 214 through a second port proximal an end 205. For illustration purposes, the rod 214 is shown in a first position as the rotation handle 206 is in a first position. However, the rod 214 can be rotated along a longitudinal axis A by moving the rotation handle 206 from a first position to a second position as shown in phantom in FIG. 2B. Rotating the rod 214 can direct blood flow to a second dialyzer.

FIG. 2C is a diagram illustrating the second embodiment of the wearable ultrafiltration device when the rod 214 is in a first position. As can be seen, in this configuration, a fluid flow path is created to direct blood flow to a dialyzer 216 and to prevent blood flow to dialyzer 218. In particular, blood reaches the port 202 where it can travel along the chamfered edge 208 and along the flow path 210 to the dialyzer 216. Then the blood will travel along the path 212 back to the valve 200 and along the chamfered edge 215 of the rod 214, where the blood will leave the valve 200 via the port 204. FIG. 2D is a diagram illustrating the second embodiment of the wearable ultrafiltration device when the rod is in a second position. As can be seen, in this configuration, a fluid flow path is created to direct blood flow to a dialyzer 218 and to prevent blood flow to dialyzer 216. In particular, blood can reach the port 202, travel along the chamfered edge 208, which is now oriented in a different position. The blood will then travel along the flow path 220 to the dialyzer 218. After filtration by the dialyzer 218, the blood will travel along the path 222 back to the valve 200 and along the chamfered edge 215 where the blood will leave the valve 200 via the port 204.

It should be noted that in this embodiment, a patient or the patient's caretaker or other user can control the position of the rod 214 and the rotation handle 206. Therefore, such a person can control the fluid path and the dialyzer which is filtering the patient's blood.

The 4 flow-interrupters shown as pinch valves in FIG. 1 can be realized in a valve having a single rod in which flow paths are cut, encased in a close-fitting tube equipped with 6 ports closely coordinated to the cuts in the rod. The cuts in the rod direct an inflow to, and an outflow from, a primary processor (ultrafilter). When the rod is rotated the inflow to the primary processor is completely transferred to the successor processor and the connection of the outflow tube is connected to the outflow port of the successor processor, leaving the primary processor isolated from the continuing flow of blood through the system. A disposable blood circuit has multiple selectable blood circuit paths each has a pre-attached ultrafilter. The blood circuit has arterial and venous connectors for connection to a patient access, the arterial and venous connectors being connected to selectable branch portions to permit the flow of blood from the arterial connector, through a selected one of the selectable blood circuit paths and then to the venous connector. A harness has a peristaltic blood pump and switch actuators. The blood circuit is configured to be received by the blood pump and switch actuators to permit blood to be pumped through a selected one of the multiple selectable blood circuit paths. The switch actuators engage the selectable branch portions to permit the selection of either of the multiple selectable blood circuit paths.

The ultrafilters may include microtubular membrane filters of a type used for hemofiltration. The apparatus may include a pre-attached waste container attached to waste ports of the ultrafilter by a common outlet. The waste may be collected without the need for a waste pump. According to additional embodiments, the disclosed subject matter includes a method of providing ambulatory ultrafiltration to a patient. The method includes fitting apparatus as in any of the above claims to a patient, ultrafiltering said patient between dialytic treatments. The ultrafiltering includes passing the patient's blood through a first of said selectable blood circuit paths, detecting the fouling of an ultrafilter in said first of said selectable blood circuit paths, closing first of said selectable blood circuit paths using said switch actuators, and replacing a fouled ultrafilter with a new ultrafilter. The switch actuators may be used to open flow through a second of said selectable blood circuit paths to permit flow of blood through an unfouled ultrafilter.

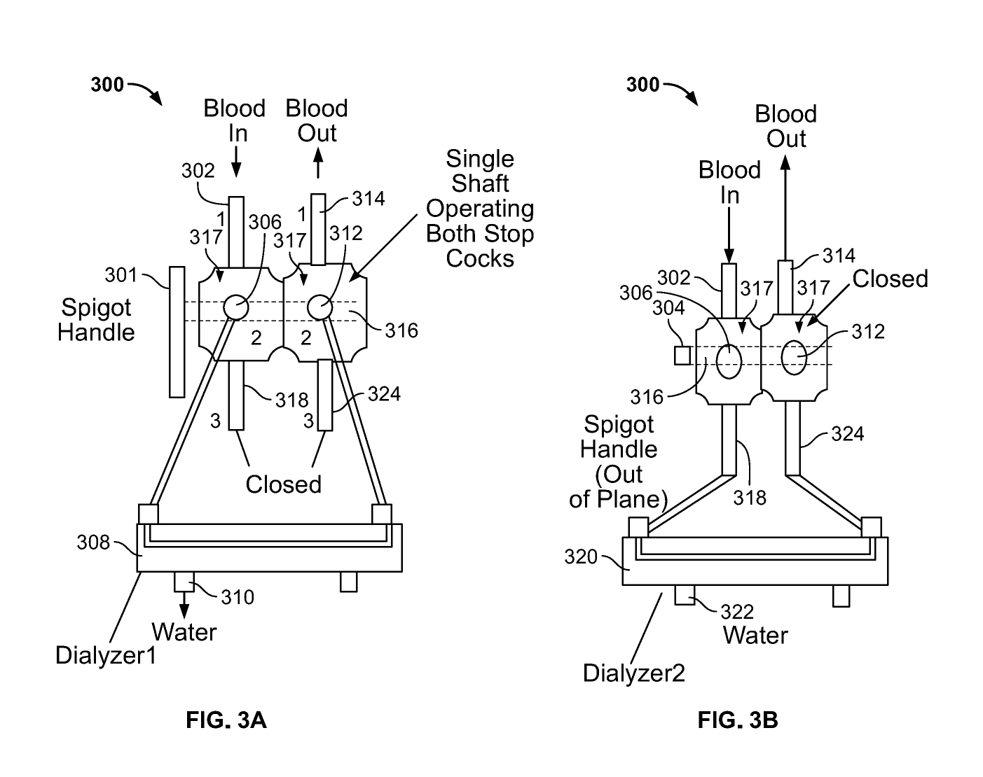

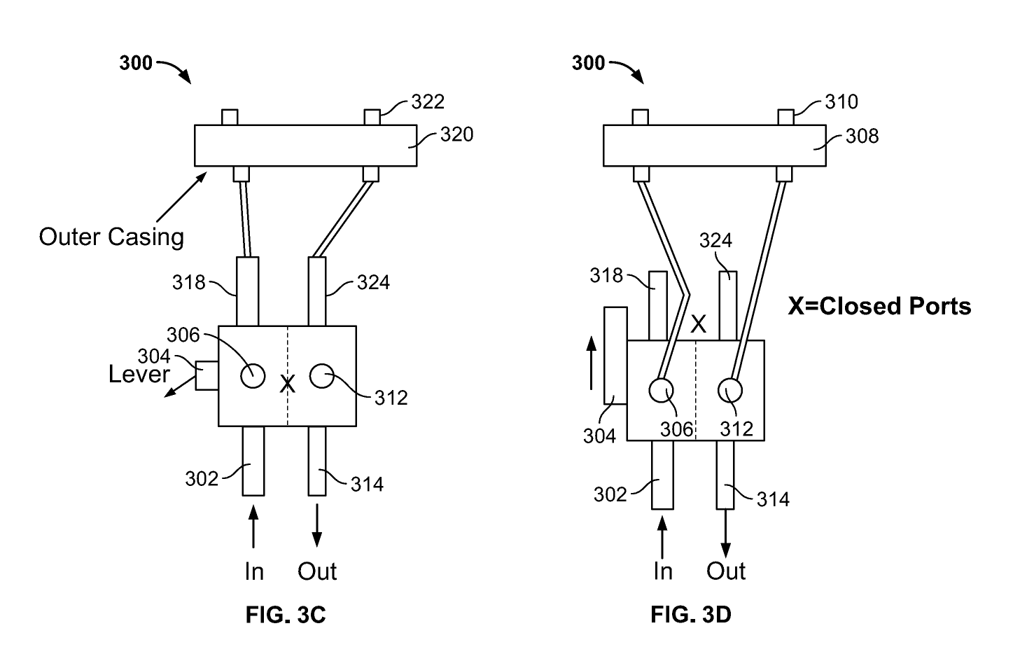

FIGS. 3A-3D illustrate another embodiment of the present invention. FIG. 3A shows a first configuration of a wearable dialyzer 300. The wearable dialyzer 300 has an input channel 302 where a patient's blood enters the wearable dialyzer 300. When a spigot handle 304 is in a first position (as shown in FIG. 3A), blood flows from a port 306 to a first dialyzer 308. Water exits the first dialyzer 308 from a port 310. Blood exits the first dialyzer 308 to the port 312. Blood then flows from the port 312 to the output channel 314 where it exits the wearable dialyzer 300 and is returned to the patient. The handle 304 is in mechanical communication with a shaft 316, which simultaneously controls operation of the two stop cocks 317 illustrated in FIG. 3A. As shown in FIG. 3A, the handle 304 is in a first position which opens the stop cocks' 317 to allow blood to flow through ports 306 and 312.

FIG. 3B shows a second configuration of the wearable dialyzer 300. In this configuration, the blood enters the wearable dialyzer 300 through the input channel 302. The spigot handle 304 is now in a second position which operates the shaft 316 and the stop cocks 317 to prevent blood from flowing through ports 306 and 312. Instead, blood will now flow to a channel 318 which directs blood to a second dialyzer 320. When the first dialyzer 308 requires service or replacement, the handle 304 can be operated from the first position shown in FIG. 3A to the second position shown in FIG. 3B to direct blood flow to the second dialyzer 320. This permits no interruption-free for dialysis of a patient. Water exits the second dialyzer 320 through the port 322. Blood flows from the second dialyzer 320 to a channel 324. When the handle 304 is in a second position, port 312 is closed which allows blood to exit the dialyzer 300 and return to the patient by flowing from channel 324 to channel 314.

FIGS. 3C and 3D show another embodiment of the wearable dialyzer 300, being operated with four-way stopcock such that the handle 304 operates the four-way stopcock. FIG. 3C illustrates the wearable dialyzer 300 in a first configuration and the handle 304 in a first position. FIG. 3D illustrates the wearable dialyzer 300 in a second configuration and the handle 304 in a second position. With reference to FIG. 3C, blood enters the wearable dialyzer 300: through the input channel 302. The spigot handle 304 is in a first position which operates the shaft 316 and the four-way stopcock to prevent blood from flowing through ports 306 and 312. Blood flows to a channel 318 which directs blood to the second dialyzer 320. Water exits the second dialyzer 320 through the port 322. Blood flows from the second dialyzer 320 to a channel 324. When the handle 304 is in the first position, port 312 is closed, which allows blood to exit the dialyzer 300 by flowing from channel 324 to channel 314, and returned to the patient. With reference to FIG. 3D, blood enters the dialyzer 300 through the input channel 302. When the spigot handle 304 is in a second position, blood flows from the port 306 to the first dialyzer 308. Water exits the first dialyzer 308 from the port 310. Blood exits the first dialyzer 308 to the port 312. Blood flows to the output channel 314 where it exits the wearable dialyzer 300, and returns to the patient.

It should be noted that in this embodiment, a patient or the patient's caretaker or other user can control the position of the handle 304. Therefore, such a person can control the fluid path and the dialyzer which is filtering the patient's blood.

All embodiments of the ultrafiltration apparatus can have an alarm or alert system for notifying the user, wearer, doctor, caretaker, etc. that it can be time to alter the device to direct blood flow from the currently used dialyzer to other dialyzer. The ultrafiltration device can send a notification that it is time to switch from one dialyzer to another. The ultrafiltration device can also send a notification that the system should be turned off due to an air bubble. Alternatively, the device can shut down automatically when detecting an air bubble. The ultrafiltration device can also send a notification that the battery is running low and provide the time remaining for the battery or percentage of battery life remaining. Finally, the ultrafiltration device can send a notification that the pump is not working properly. The ultrafiltration device can have hardware to connect to the internet to send a notification via text message, email, etc. on any type of device such as a computer, PC, tablet, phone, etc.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

What is claimed is:

1. A wearable ultrafiltration apparatus, comprising:
a first dialyzer for filtering a patient's blood along a first fluid path;
a second dialyzer for filtering the patient's blood along a second fluid path; and
a valve being positionable in a first position for directing the patient's blood along the first fluid path and being positionable in a second position for directing the patient's blood along the second fluid path;
wherein when the valve is in the first position, blood flows along the first fluid path and prevents blood from flowing along the second fluid path and when the valve is in the second position, blood flows along the second fluid path and prevents blood from flowing along the first fluid path;
wherein when the valve is in the first position, the second dialyzer is idle and capable of being serviced or replaced, and when the valve is in the second position, the first dialyzer is idle and capable of being serviced or replaced.

2. The apparatus of claim 1, wherein the valve includes a first pinch valve for directing blood along the first fluid path and a second pinch valve for directing blood along a second fluid path.

3. The apparatus of claim 2, wherein the first fluid path and the second fluid path converge at a Y connector before the patient's blood is returned to the patient.

4. The apparatus of claim 3, wherein the first fluid path includes a third pinch valve for directing blood flow to the Y connector and the second fluid path includes a fourth pinch valve for directing blood flow to the Y connector.

5. The apparatus of claim 4, wherein when the valve is in the first position, the first and third pinch valves are in an open position to direct blood flow along the first fluid path and the second and fourth pinch valves are in a closed position to prevent blood flow along the second fluid path.

6. The apparatus of claim 5, wherein when the valve is in the second position, the second and fourth pinch valves are in an open position for directing blood flow along the second fluid path and the first and third pinch valves are in a closed position for preventing blood flow along the first fluid path.

7. The apparatus of claim 6, wherein the first dialyzer includes a first port for filtrate water disposal and the second dialyzer includes a second port for filtrate water disposal.

8. The apparatus of claim 7, wherein a battery operated pump directs the patient's blood to the apparatus.

9. The apparatus of claim 1, wherein the first dialyzer and the second dialyzer are microtubular membrane filters of a type used for hemofiltration.

10. The apparatus of claim 8, further comprising a waste container attached to the first port and the second port by a common outlet.

11. The apparatus of claim 1, wherein waste is collected without the need for a waste pump.

12. The apparatus of claim 1, wherein the valve includes a rod.

13. The apparatus of claim 12, wherein the rod includes a handle attached thereto for rotating the rod from the first position to the second position.

14. The apparatus of claim 13, wherein the rod includes a first edge positioned in a first orientation when the rod is in the first position, the first edge directing the patient's blood to the first dialyzer along the first fluid path.

15. The apparatus of claim 14, wherein when the rod is in the second position, the first edge is positioned in a second orientation for directing the patient's blood to the second dialyzer along the second fluid path.

16. The apparatus of claim 15, wherein the rod includes a second edge for receiving the blood from the first dialyzer when the rod is in the first position or the second dialyzer when the rod is in the second position.

17. The apparatus of claim 16, wherein the rod is rotated along a longitudinal axis of the rod.

18. The apparatus of claim 1, wherein the valve comprises a spigot handle having a shaft operable to a first position and a second position.

19. The apparatus of claim 18, wherein the first fluid path includes a first port for directing blood to the first dialyzer and a second port for receiving blood from the first dialyzer.

20. The apparatus of claim 19, wherein the second fluid path includes a first channel for directing blood to the second dialyzer and a second channel for receiving blood from the second dialyzer.

21. The apparatus of claim 20, wherein when the spigot handle is in the first portion, blood flows through the first fluid path and when the spigot handle is in the second position, blood flows through the second fluid path.

22. The apparatus of claim 1, wherein the patient can position the valve from the first position to the second position.

23. The apparatus of claim 4, wherein the patient can position at least one of the first pinch valve, the second pinch valve, the third pinch valve, or the fourth pinch valve.

24. The apparatus of claim 13, wherein the patient can use the handle to position the rod in the first position and the second position.

25. The apparatus of claim 18, wherein the patient can position the spigot handle in the first position and the second position.

26. A method of providing ambulatory ultrafiltration to a patient, comprising:
passing a patient's blood through a first fluid path of a wearable ultrafiltration device;
detecting a fouling of a dialyzer in the first fluid path;
closing the first fluid path using a valve of the wearable ultrafiltration device; a
opening a second fluid path of the wearable ultrafiltration device to permit flow of blood through a second dialyzer.

27. The method of claim 26, further comprising replacing the fouled dialyzer in the first fluid path with a new dialyzer.
